(19) United States
(12) Patent Application Publication
(10) Pub. No.: US 2020/0354795 A1
(43) Pub. Date: Nov. 12, 2020

(54) METHOD FOR DIAGNOSING PANCREATIC CANCER VIA BACTERIAL METAGENOMIC ANALYSIS

(71) Applicants: MD HEALTHCARE INC., Seoul (KR); SEOUL NATIONAL UNIVERSITY R & DB FOUNDATION, Seoul (KR)

(72) Inventors: Yoon-Keun Kim, Gyeonggi-do (KR); Taesung Park, Seoul (KR); Jin-Young Jang, Seoul (KR)

(21) Appl. No.: 16/629,348

(22) PCT Filed: Dec. 21, 2017
(86) PCT No.: PCT/KR2017/015174

§ 371 Date: Jan. 8, 2020

(30) Foreign Application Priority Data
Dec. 26, 2016 (KR) 10-2016-0179234
Dec. 20, 2017 (KR) 10-2017-0175821

(51) Int. Cl. C12Q 1/6886; C12Q 1/689

(52) U.S. Cl. CPC C12Q 1/6886; C12Q 1/689

(57) ABSTRACT

The present invention relates to a method of diagnosing pancreatic cancer through bacterial metagenomic analysis, and more particularly to a method of diagnosing pancreatic cancer by analyzing an increase or decrease in content of specific bacteria-derived extracellular vesicles through metagenomic analysis using a subject-derived sample. Extracellular vesicles secreted from bacteria existing in the environment are absorbed into the human body, and thus may directly affect the occurrence of cancer, and it is difficult to diagnose pancreatic cancer early before the onset of symptoms so that efficient treatment thereof is difficult. Thus, according to the present invention, a risk for pancreatic cancer can be predicted through metagenomic analysis of bacteria-derived extracellular vesicles using a human body-derived sample, and thus a risk group of pancreatic cancer can be diagnosed early and predicted, thereby delaying the onset of pancreatic cancer or preventing pancreatic cancer through appropriate management, and even after pancreatic cancer occurs, early diagnosis for pancreatic cancer can be implemented, thereby lowering the incidence of pancreatic cancer and increasing therapeutic effects.

### TECHNICAL FIELD

The present invention relates to a method of diagnosing pancreatic cancer through bacterial metagenomic analysis, and more particularly to a method of diagnosing pancreatic cancer by analyzing an increase or decrease in content of extracellular vesicles derived from specific bacteria by bacterial metagenomic analysis using a subject-derived sample.

### BACKGROUND ART

Pancreatic cancer is a malignant tumor originating from the pancreas, which has a 5-year survival rate of less than 10% despite advances in modern medicine. The reason why pancreatic cancer has a 5-year survival rate of less than 10% despite advances in modern medicine is because pancreatic cancer is found in an advanced state. To address this problem, it is efficient to provide a method for preventing the onset of pancreatic cancer in a high risk group based on causative factors of pancreatic cancer.

Although pancreatic cancer is the 8th most common cancer in Korea, it follows lung cancer, liver cancer, gastric cancer, and colorectal cancer as the cause of cancer death. Although no clear cause of pancreatic cancer has yet been identified, smoking is considered as a risk factor for pancreatic cancer like lung cancer and esophageal cancer, and it is reported that smokers are about 2 to 3 times more likely to develop pancreatic cancer than nonsmokers. In addition to smoking, diseases such as chronic pancreatitis, obesity, and diabetes, high-fat, high-calorie diets, drinking, and the like are known to increase the risk of developing pancreatic cancer. Although genetic factors also affect the onset of pancreatic cancer, hereditary pancreatic cancer is very rare in Korea.

The symptoms of pancreatic cancer are non-specific, and symptoms that can be found in various pancreatic diseases may occur, abdominal pain, anorexia, weight loss, jaundice, and the like are the most common symptoms, abdominal pain and weight loss occur in most pancreatic cancer patients, and jaundice occurs in most patients with pancreatic head cancer. Cancers that occur in the body and tail sites of the pancreas are rarely symptomatic at an early stage, and thus are often found later.

To date, there is no certified screening method capable of detecting pancreatic cancer early before the onset of symptoms, and although research on abdominal ultrasonography, abdominal computed tomography (CT), magnetic resonance imaging (MRI), endoscopic retrograde cholangiopancreatography (ERCP), endoscopic ultrasound (EUS), proton emission tomography (PET), and serum tumor marker (CA19-9) testing is actively conducted, no valid diagnostic method has yet been proposed. Therefore, there is an urgent need to develop a method for early diagnosis of pancreatic cancer and capable of enhancing therapeutic efficiency, and since it is very important to differentiate early diagnosis and responses to treatment by predicting the onset of pancreatic cancer, research and technology development are required in advance.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, is analytics for metagenomic data obtained from samples collected from the environment, and refers collectively to the total of genomes of all microbiomes in the natural environment and was first used in 1998 by Jo Handelsman (Handelsman et al., 1998 Chem. Biol. 5, R245-249). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of pancreatic cancer, identification of causative factors of pancreatic cancer through metagenomic analysis of bacteria-derived vesicles isolated from a human-derived substance, such as blood and the like, and a method of diagnosing pancreatic cancer have never been reported.

### DISCLOSURE

### Technical Problem

To diagnose the causative factors and risk of pancreatic cancer, the inventors of the present invention extracted a gene from bacteria-derived extracellular vesicles present in blood, which is a subject-derived sample and performed metagenomic analysis thereon, and consequently, identified bacteria-derived extracellular vesicles capable of acting as a causative factor of pancreatic cancer, and thus completed the present invention based on this finding.

Therefore, an object of the present invention is to provide a method of providing information for pancreatic cancer diagnosis through metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

According to an aspect of the present invention, there is provided a method of providing information for pancreatic cancer diagnosis, comprising the following processes:

(a) extracting DNA from extracellular vesicles isolated from a subject sample;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject sample with that of a normal individual-derived sample through sequencing of a product of the PCR.

The present invention also provides a method of diagnosing pancreatic cancer, comprising the following processes:

(a) extracting DNA from extracellular vesicles isolated from a subject sample;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject sample with that of a normal individual-derived sample through sequencing of a product of the PCR.

The present invention also provides a method of predicting a risk for pancreatic cancer, comprising the following processes:

(a) extracting DNA from extracellular vesicles isolated from a subject sample;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject sample with that of a normal individual-derived sample through sequencing of a product of the PCR.

In one embodiment of the present invention, process (c) may comprise comparing an increase or decrease in content of extracellular vesicles derived from one or more bacteria selected from the group consisting of the phylum Fusobacteria, the phylum Thermi, the phylum Cyanobacteria, the phylum Verrucomicrobia, the phylum Deferribacteres, the phylum Armatimonadetes, and the phylum Euryarchaeota.

In one embodiment of the present invention, process (c) may comprise comparing an increase or decrease in content of extracellular vesicles derived from one or more bacteria selected from the group consisting of the class Erysipelotrichi, the class Betaproteobacteria, the class Deltaproteobacteria, the class Chloroplast, the class Verrucomicrobiae, the class Deferribacteres, the class Fimbriimonadia, and the class Halobacteria.

In one embodiment of the present invention, process (c) may comprise comparing an increase or decrease in content of extracellular vesicles derived from one or more bacteria selected from the group consisting of the order Erysipelotrichales, the order Rhizobiales, the order Burkholderiales, the order Fusobacteriales, the order Deinococcales, the order Rhodobacterales, the order Bifidobacteriales, the order Flavobacteriales, the order Streptophyta, the order Verrucomicrobiales, the order Rickettsiales, the order Deferribacterales, the order Fimbriimonadales, the order Oceanospirillales, the order Anaeroplasmatales, the order Halobacteriales, the order RF32, and the order Bdellovibrionales.

In one embodiment of the present invention, process (c) may comprise comparing an increase or decrease in content of extracellular vesicles derived from one or more bacteria selected from the group consisting of the family Rhizobiaceae, the family Oxalobacteraceae, the family Rikenellaceae, the family Erysipelotrichaceae, the family S24-7, the family Comamonadaceae, the family Pseudomonadaceae, the family Rhodobacteraceae, the family Methylobacteriaceae, the family Clostridiaceae, the family Bifidobacteriaceae, the family Aerococcaceae, the family Weeksellaceae, the family Veillonellaceae, the family Carnobacteriaceae, the family Planococcaceae, the family Prevotellaceae, the family Verrucomicrobiaceae, the family mitochondria, the family Deferribacteraceae, the family Peptococcaceae, the family Fimbriimonadaceae, the family Christensenellaceae, the family Halomonadaceae, the family Gordoniaceae, the family Pseudonocardiaceae, and the family Bdellovibrionaceae.

In one embodiment of the present invention, process (c) may comprise comparing an increase or decrease in content of extracellular vesicles derived from one or more bacteria selected from the group consisting of the genus Catenibacterium, the genus Geobacillus, the genus Cloacibacterium, the genus Faecalibacterium, the genus Pseudomonas, the genus Methylobacterium, the genus Prevotella, the genus Paracoccus, the genus Enhydrobacter, the genus Bifidobacterium, the genus Haemophilus, the genus Micrococcus, the genus Lactococcus, the genus Oscillospira, the genus Dorea, the genus Akkermansia, the genus Mucispirillum, the genus Fimbriimonas, the genus Enterobacter, the genus Gordonia, the genus Chromohalobacter, the genus Pseudonocardia, the genus Halobacterium, and the genus Bdellovibrio.

In one embodiment of the present invention, the subject samples may be blood. In one embodiment of the present invention, the blood may be whole blood, serum, plasma, or blood mononuclear cells.

### Advantageous Effects

Extracellular vesicles secreted from bacteria existing in the environment are absorbed into the human body, and thus may directly affect the occurrence of cancer, and it is difficult to diagnose pancreatic cancer early before the onset of symptoms so that efficient treatment thereof is difficult. Thus, according to the present invention, the causative factor and risk of pancreatic cancer can be diagosed through metagenomic analysis of bacteria-derived extracellular vesicles using a human body-derived sample, and thus a risk group of pancreatic cancer can be diagnosed early, thereby delaying the onset of pancreatic cancer or preventing pancreatic cancer through appropriate management, and even after pancreatic cancer occurs, early diagnosis for pancreatic cancer can be implemented, thereby lowering the incidence of pancreatic cancer and increasing therapeutic effects. In addition, the metagenomic analysis enables patients diagnosed with pancreatic cancer to avoid exposure to causative factors predicted thereby, whereby the progression of cancer is ameliorated, or the recurrence of pancreatic cancer can be prevented.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, blood and various organs were extracted.

FIG. 2 illustrates distribution results of bacteria-derived EVs exhibiting significant diagnostic performance at a phylum level, after metagenomic analysis of bacteria-derived EVs isolated from pancreatic cancer patient-derived blood and normal individual-derived blood.

FIG. 3 illustrates distribution results of bacteria-derived EVs exhibiting significant diagnostic performance at a class level, after metagenomic analysis of bacteria-derived EVs isolated from pancreatic cancer patient-derived blood and normal individual-derived blood.

FIG. 4 illustrates distribution results of bacteria-derived EVs exhibiting significant diagnostic performance at an order level, after metagenomic analysis of bacteria-derived EVs isolated from pancreatic cancer patient-derived blood and normal individual-derived blood.

FIG. 5 illustrates distribution results of bacteria-derived EVs exhibiting significant diagnostic performance at a family level, after metagenomic analysis of bacteria-derived EVs isolated from pancreatic cancer patient-derived blood and normal individual-derived blood.

FIG. 6 illustrates distribution results of bacteria-derived EVs exhibiting significant diagnostic performance at a genus level, after metagenomic analysis of bacteria-derived EVs isolated from pancreatic cancer patient-derived blood and normal individual-derived blood.

### BEST MODE

The present invention relates to a method of diagnosing pancreatic cancer through bacterial metagenomic analysis. The inventors of the present invention extracted genes from bacteria-derived extracellular vesicles using a subject-derived sample, performed metagenomic analysis thereon, and identified bacteria-derived extracellular vesicles capable of acting as a causative factor of pancreatic cancer.

Therefore, the present invention provides a method of providing information for diagnosing pancreatic cancer, the method comprising:

(a) extracting DNA from extracellular vesicles isolated from a subject sample;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject sample with that of a normal individual-derived sample through sequencing of a product of the PCR.

The term “pancreatic cancer diagnosis” as used herein refers to determining whether a patient has a risk for pancreatic cancer, whether the risk for pancreatic cancer is relatively high, or whether pancreatic cancer has already occurred. The method of the present invention may be used to delay the onset of pancreatic cancer through special and appropriate care for a specific patient, which is a patient having a high risk for pancreatic cancer or prevent the onset of pancreatic cancer. In addition, the method may be clinically used to determine treatment by selecting the most appropriate treatment method through early diagnosis of pancreatic cancer.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

In the present invention, the subject sample may be blood or urine, and the blood may be whole blood, serum, plasma, or blood mononuclear cells, but the present invention is not limited thereto.

In an embodiment of the present invention, metagenomic analysis is performed on the bacteria- and archaea-derived extracellular vesicles, and bacteria-derived extracellular vesicles capable of acting as a cause of the onset of pancreatic cancer were actually identified by analysis at phylum, class, order, family, and genus levels.

More particularly, in one embodiment of the present invention, as a result of performing bacterial metagenomic analysis on extracellular vesicles present in subject-derived blood samples at a phylum level, the content of extracellular vesicles derived from bacteria belonging to the phylum Fusobacteria, the phylum Thermi, the phylum Cyanobacteria, the phylum Verrucomicrobia, the phylum Deferribacteres, the phylum Armatimonadetes, and the phylum Euryarchaeota was significantly different between pancreatic cancer patients and normal individuals (see Example 4).

More particularly, in one embodiment of the present invention, as a result of performing bacterial metagenomic analysis on extracellular vesicles present in subject-derived blood samples at a class level, the content of extracellular vesicles derived from bacteria belonging to the class Erysipelotrichi, the class Betaproteobacteria, the class Deltaproteobacteria, the class Chloroplast, the class Verrucomicrobiae, the class Deferribacteres, the class Fimbriimonadia, and the class Halobacteria was significantly different between pancreatic cancer patients and normal individuals (see Example 4).

More particularly, in one embodiment of the present invention, as a result of performing bacterial metagenomic analysis on extracellular vesicles present in subject-derived blood samples at an order level, the content of extracellular vesicles derived from bacteria belonging to the order Erysipelotrichales, the order Rhizobiales, the order Burkholderiales, the order Fusobacteriales, the order Deinococcales, the order Rhodobacterales, the order Bifidobacteriales, the order Flavobacteriales, the order Streptophyta, the order Verrucomicrobiales, the order Rickettsiales, the order Deferribacterales, the order Fimbriimonadales, the order Oceanospirillales, the order Anaeroplasmatales, the order Halobacteriales, the order RF32, and the order Bdellovibrionales was significantly different between pancreatic cancer patients and normal individuals (see Example 4).

More particularly, in one embodiment of the present invention, as a result of performing bacterial metagenomic analysis on extracellular vesicles present in subject-derived blood samples at a family level, the content of extracellular vesicles derived from bacteria belonging to the family Rhizobiaceae, the family Oxalobacteraceae, the family Rikenellaceae, the family Erysipelotrichaceae, the family S24-7, the family Comamonadaceae, the family Pseudomonadaceae, the family Rhodobacteraceae, the family Methylobacteriaceae, the family Clostridiaceae, the family Bifidobacteriaceae, the family Aerococcaceae, the family Weeksellaceae, the family Veillonellaceae, the family Carnobacteriaceae, the family Planococcaceae, the family Prevotellaceae, the family Verrucomicrobiaceae, the family mitochondria, the family Deferribacteraceae, the family Peptococcaceae, the family Fimbriimonadaceae, the family Christensenellaceae, the family Halomonadaceae, the family Gordoniaceae, the family Pseudonocardiaceae, and the family Bdellovibrionaceae was significantly different between pancreatic cancer patients and normal individuals (see Example 4).

More particularly, in one embodiment of the present invention, as a result of performing bacterial metagenomic analysis on extracellular vesicles present in subject-derived blood samples at a genus level, the content of extracellular vesicles derived from bacteria belonging to the genus Catenibacterium, the genus Geobacillus, the genus Cloacibacterium, the genus Faecalibacterium, the genus Pseudomonas, the genus Methylobacterium, the genus Prevotella, the genus Paracoccus, the genus Enhydrobacter, the genus Bifidobacterium, the genus Haemophilus, the genus Micrococcus, the genus Lactococcus, the genus Oscillospira, the genus Dorea, the genus Akkermansia, the genus Mucispirillum, the genus Fimbriimonas, the genus Enterobacter, the genus Gordonia, the genus Chromohalobacter, the genus Pseudonocardia, the genus Halobacterium, and the genus Bdellovibrio was significantly different between pancreatic cancer patients and normal individuals (see Example 4).

Through the results of the examples, it was confirmed that distribution variables of the identified bacteria-derived extracellular vesicles could be usefully used for the prediction of the onset of pancreatic cancer.

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Intestinal Bacteria and Bacteria-Derived Extracellular Vesicles

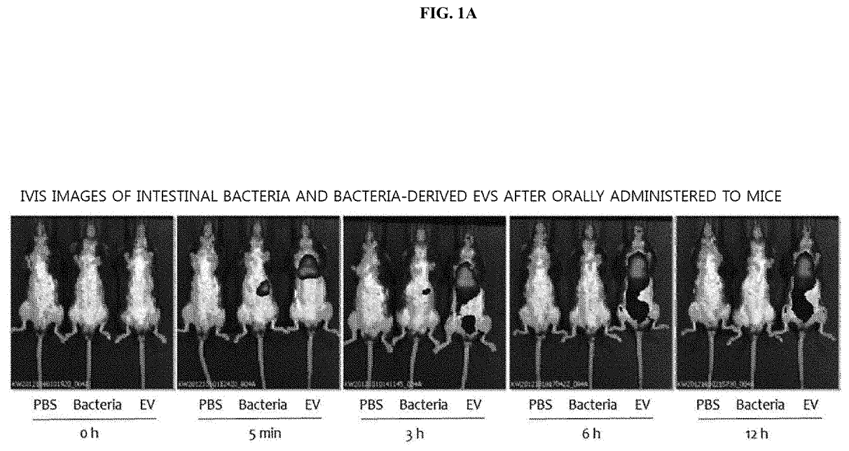

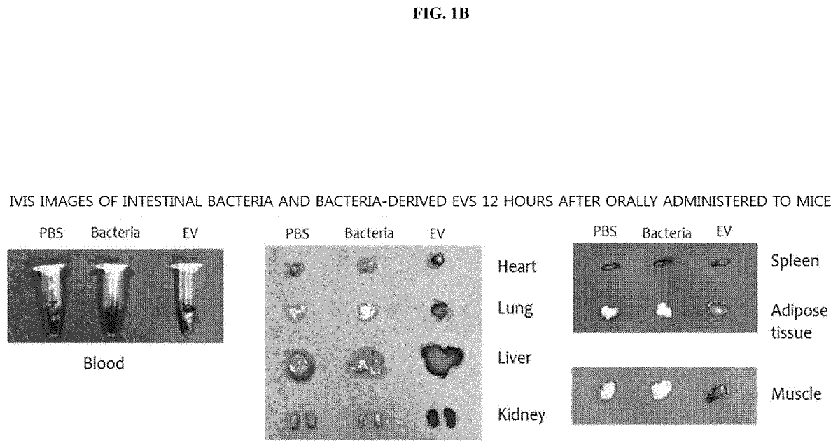

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction from Blood

To isolate extracellular vesicles and extract DNA, from blood, first, blood was added to a 10 ml tube and centrifuged at 3,500×g and 4 □ for 10 min to precipitate a suspension, and only a supernatant was collected, which was then placed in a new 10 ml tube. The collected supernatant was filtered using a 0.22 μm filter to remove bacteria and impurities, and then placed in centrifugal filters (50 kD) and centrifuged at 1500×g and 4 □ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4 □ for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the blood according to the above-described method was boiled at 100 □ to allow the internal DNA to come out of the lipid and then cooled on ice. Next, the resulting vesicles were centrifuged at 10,000×g and 4 □ for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3. Metagenomic Analysis Using DNA Extracted from Blood

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score<20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Pancreatic Cancer Diagnostic Model Based on Metagenomic Analysis of Bacteria-Derived EVs Isolated from Blood

EVs were isolated from blood samples of 176 pancreatic cancer patients and 271 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

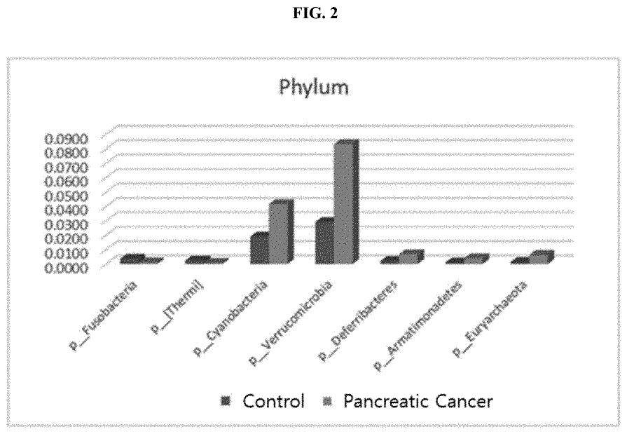

As a result of analyzing bacteria-derived EVs in blood at a phylum level, a diagnostic model developed using bacteria belonging to the phylum Fusobacteria, the phylum Thermi, the phylum Cyanobacteria, the phylum Verrucomicrobia, the phylum Deferribacteres, the phylum Armatimonadetes, and the phylum Euryarchaeota as a biomarker exhibited significant diagnostic performance for pancreatic cancer (see Table 2 and FIG. 2).

TABLE 2

| | | | Pancreatic | | t-test | | | | | | | |
|---|---|---|---|---|---|---|---|---|---|---|---|---|
| | Control | | Cancer | | p- | | Training Set | | | Test Set | | |
| Taxon | Mean | SD | Mean | SD | value | Ratio | AUC | sensitivity | specificity | AUC | sensitivity | specificity |
| p_Fusobacteria | 0.0035 | 0.0102 | 0.0011 | 0.0021 | 0.0001 | 0.31 | 0.60 | 0.66 | 0.02 | 0.56 | 0.99 | 0.00 |
| p_[Thermi] | 0.0024 | 0.0060 | 0.0008 | 0.0015 | 0.0000 | 0.32 | 0.63 | 0.67 | 0.46 | 0.52 | 0.62 | 0.41 |
| p_Cyanobacteria | 0.0190 | 0.0455 | 0.0415 | 0.0799 | 0.0007 | 2.19 | 0.68 | 0.95 | 0.87 | 0.69 | 0.98 | 0.12 |
| p_Verrucomicrobia | 0.0291 | 0.0443 | 0.0832 | 0.0517 | 0.0000 | 2.86 | 0.83 | 0.90 | 0.54 | 0.90 | 0.94 | 0.45 |
| p_Deferribacteres | 0.0020 | 0.0067 | 0.0067 | 0.0068 | 0.0000 | 3.39 | 0.79 | 0.88 | 0.39 | 0.82 | 0.93 | 0.37 |
| p_Armatimonadetes | 0.0010 | 0.0041 | 0.0039 | 0.0072 | 0.0000 | 3.93 | 0.63 | 0.95 | 0.20 | 0.60 | 0.95 | 0.27 |
| p_Euryarchaeota | 0.0013 | 0.0035 | 0.0062 | 0.0150 | 0.0000 | 4.84 | 0.68 | 0.91 | 0.23 | 0.70 | 0.92 | 0.27 |

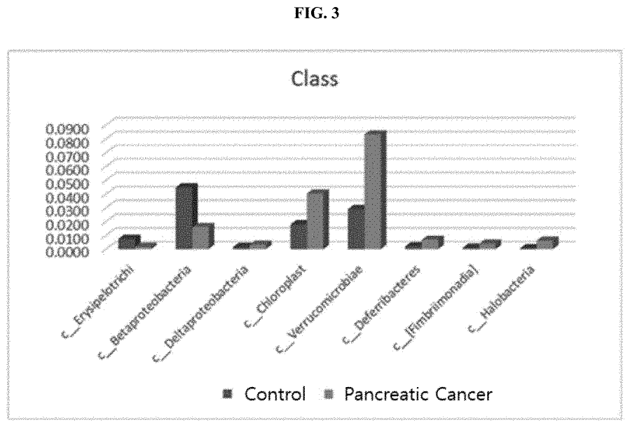

As a result of analyzing bacteria-derived EVs in blood at a class level, a diagnostic model developed using bacteria belonging to the class Erysipelotrichi, the class Betaproteobacteria, the class Deltaproteobacteria, the class Chloroplast, the class Verrucomicrobiae, the class Deferribacteres, the class Fimbriimonadia, and the class Halobacteria as a biomarker exhibited significant diagnostic performance for pancreatic cancer (see Table 3 and FIG. 3).

TABLE 3

| | | | Pancreatic | | t-test | | | | | | | |
|---|---|---|---|---|---|---|---|---|---|---|---|---|
| | Control | | Cancer | | p- | | Training Set | | | Test Set | | |
| Taxon | Mean | SD | Mean | SD | value | Ratio | AUC | sensitivity | specificity | AUC | sensitivity | specificity |
| c_Erysipelotrichi | 0.0074 | 0.0160 | 0.0015 | 0.0023 | 0.0000 | 0.21 | 0.63 | 0.82 | 0.24 | 0.63 | 0.75 | 0.28 |
| c_Betaproteobacteria | 0.0447 | 0.0553 | 0.0159 | 0.0277 | 0.0000 | 0.35 | 0.82 | 0.78 | 0.64 | 0.71 | 0.68 | 0.53 |
| c_Deltaprotebacteria | 0.0015 | 0.0032 | 0.0030 | 0.0050 | 0.0003 | 2.03 | 0.57 | 0.95 | 0.16 | 0.55 | 0.96 | 0.17 |
| c_Chloroplast | 0.0179 | 0.0450 | 0.0403 | 0.0791 | 0.0007 | 2.25 | 0.69 | 0.94 | 0.14 | 0.75 | 0.96 | 0.10 |
| c_Verrucomicrobiae | 0.0289 | 0.0443 | 0.0831 | 0.0516 | 0.0000 | 2.87 | 0.86 | 0.92 | 0.60 | 0.81 | 0.87 | 0.60 |
| c_Deferribacteres | 0.0020 | 0.0067 | 0.0067 | 0.0068 | 0.0000 | 3.39 | 0.81 | 0.94 | 0.35 | 0.75 | 0.90 | 0.28 |
| c_[Fimbriimonadia] | 0.0010 | 0.0041 | 0.0039 | 0.0072 | 0.0000 | 3.94 | 0.60 | 0.95 | 0.17 | 0.66 | 0.99 | 0.17 |
| c_Halobacteria | 0.0005 | 0.0021 | 0.0060 | 0.0150 | 0.0000 | 11.88 | 0.75 | 0.94 | 0.31 | 0.77 | 0.99 | 0.33 |

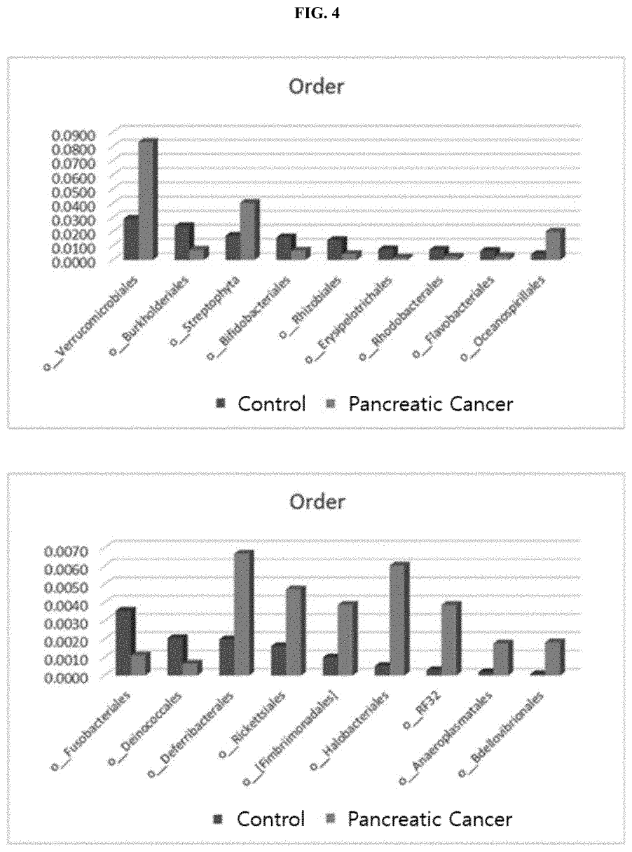

As a result of analyzing bacteria-derived EVs in blood at an order level, a diagnostic model developed using bacteria belonging to the order Erysipelotrichales, the order Rhizobiales, the order Burkholderiales, the order Fusobacteriales, the order Deinococcales, the order Rhodobacterales, the order Bifidobacteriales, the order Flavobacteriales, the order Streptophyta, the order Verrucomicrobiales, the order Rickettsiales, the order Deferribacterales, the order Fimbriimonadales, the order Oceanospirillales, the order Anaeroplasmatales, the order Halobacteriales, the order RF32, and the order Bdellovibrionales as a biomarker exhibited significant diagnostic performance for pancreatic cancer (see Table 4 and FIG. 4).

TABLE 4

| | | | Pancreatic | | t-test | | | | | | | |
|---|---|---|---|---|---|---|---|---|---|---|---|---|
| | Control | | Cancer | | p- | | Training Set | | | Test Set | | |
| Taxon | Mean | SD | Mean | SD | value | Ratio | AUC | sensitivity | specificity | AUC | sensitivity | specificity |
| o_Erysipelotrichales | 0.0074 | 0.0160 | 0.0015 | 0.0023 | 0.0000 | 0.21 | 0.60 | 0.74 | 0.37 | 0.62 | 0.60 | 0.52 |
| o_Rhizobiales | 0.0139 | 0.0172 | 0.0041 | 0.0096 | 0.0000 | 0.29 | 0.74 | 0.73 | 0.51 | 0.74 | 0.72 | 0.65 |
| o_Burkholderiales | 0.0238 | 0.0256 | 0.0072 | 0.0256 | 0.0000 | 0.30 | 0.82 | 0.77 | 0.80 | 0.82 | 0.76 | 0.67 |
| o_Fusobacteriales | 0.0035 | 0.0102 | 0.0011 | 0.0021 | 0.0001 | 0.31 | 0.58 | 0.92 | 0.11 | 0.52 | 0.98 | 0.02 |
| o_Deinococcales | 0.0020 | 0.0059 | 0.0006 | 0.0014 | 0.0002 | 0.32 | 0.50 | 0.99 | 0.00 | 0.51 | 0.98 | 0.02 |
| o_Rhodobacterales | 0.0071 | 0.0199 | 0.0023 | 0.0038 | 0.0002 | 0.33 | 0.64 | 0.99 | 0.16 | 0.68 | 0.94 | 0.13 |
| o_Bifidobacteriales | 0.0159 | 0.0206 | 0.0064 | 0.0084 | 0.0000 | 0.41 | 0.64 | 0.72 | 0.45 | 0.67 | 0.67 | 0.50 |
| o_Flavobacteriales | 0.0063 | 0.0110 | 0.0026 | 0.0098 | 0.0003 | 0.41 | 0.61 | 0.71 | 0.46 | 0.66 | 0.63 | 0.49 |
| o_Streptophyta | 0.0170 | 0.0447 | 0.0402 | 0.0791 | 0.0004 | 2.37 | 0.68 | 0.94 | 0.16 | 0.61 | 0.98 | 0.13 |
| o_Verrucomicrobiales | 0.0289 | 0.0443 | 0.0831 | 0.0516 | 0.0000 | 2.87 | 0.84 | 0.90 | 0.54 | 0.86 | 0.89 | 0.56 |
| o_Rickettsiales | 0.0016 | 0.0055 | 0.0047 | 0.0085 | 0.0000 | 2.94 | 0.71 | 0.94 | 0.17 | 0.65 | 0.95 | 0.21 |
| o_Deferribacterales | 0.0020 | 0.0067 | 0.0067 | 0.0068 | 0.0000 | 3.39 | 0.74 | 0.94 | 0.35 | 0.73 | 0.83 | 0.52 |
| o_[Fimbriimonadales] | 0.0010 | 0.0041 | 0.0039 | 0.0072 | 0.0000 | 3.94 | 0.60 | 0.95 | 0.28 | 0.57 | 0.95 | 0.17 |
| o_Oceanospirillales | 0.0040 | 0.0079 | 0.0199 | 0.0443 | 0.0000 | 4.98 | 0.84 | 0.89 | 0.55 | 0.73 | 0.86 | 0.56 |
| o_Anaeroplasmatales | 0.0002 | 0.0010 | 0.0017 | 0.0027 | 0.0000 | 10.1 | 0.78 | 0.97 | 0.40 | 0.63 | 0.95 | 0.54 |
| o_Halobacteriales | 0.0005 | 0.0021 | 0.0060 | 0.0150 | 0.0000 | 11.8 | 0.78 | 0.95 | 0.43 | 0.67 | 0.95 | 0.31 |
| o_RF32 | 0.0003 | 0.0013 | 0.0039 | 0.0064 | 0.0000 | 13.6 | 0.77 | 0.96 | 0.43 | 0.74 | 0.94 | 0.52 |
| o_Bdellovibrionales | 0.0001 | 0.0003 | 0.0018 | 0.0036 | 0.0000 | 25.4 | 0.54 | 0.97 | 0.33 | 0.52 | 0.98 | 0.15 |

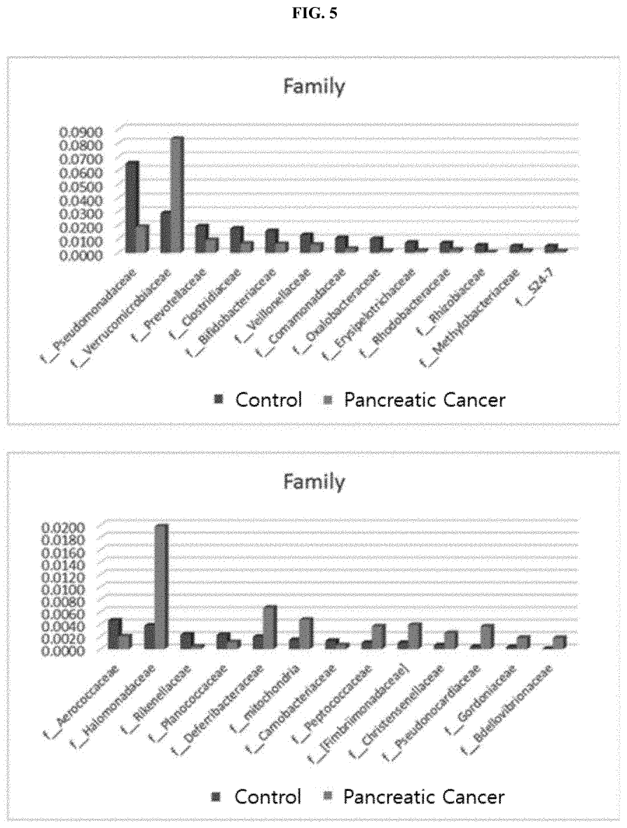

As a result of analyzing bacteria-derived EVs in blood at a family level, a diagnostic model developed using bacteria belonging to the family Rhizobiaceae, the family Oxalobacteraceae, the family Rikenellaceae, the family Erysipelotrichaceae, the family S24-7, the family Comamonadaceae, the family Pseudomonadaceae, the family Rhodobacteraceae, the family Methylobacteriaceae, the family Clostridiaceae, the family Bifidobacteriaceae, the family Aerococcaceae, the family Weeksellaceae, the family Veillonellaceae, the family Carnobacteriaceae, the family Planococcaceae, the family Prevotellaceae, the family Verrucomicrobiaceae, the family mitochondria, the family Deferribacteraceae, the family Peptococcaceae, the family Fimbriimonadaceae, the family Christensenellaceae, the family Halomonadaceae, the family Gordoniaceae, the family Pseudonocardiaceae, and the family Bdellovibrionaceae as a biomarker exhibited significant diagnostic performance for pancreatic cancer (see Table 5 and FIG. 5).

TABLE 5

| | | | Pancreatic | | t-test | | | | | | | |
|---|---|---|---|---|---|---|---|---|---|---|---|---|
| | Control | | Cancer | | p- | | Training Set | | | Test Set | | |
| Taxon | Mean | SD | Mean | SD | value | Ratio | AUC | sensitivity | specificity | AUC | sensitivity | specificity |
| f_Rhizobiaceae | 0.0054 | 0.0102 | 0.0004 | 0.0012 | 0.0000 | 0.08 | 0.71 | 0.58 | 0.74 | 0.73 | 0.52 | 0.84 |
| f_Oxalobacteraceae | 0.0101 | 0.0161 | 0.0014 | 0.0070 | 0.0000 | 0.14 | 0.79 | 0.66 | 0.88 | 0.75 | 0.60 | 0.82 |
| f_Rikenellaceae | 0.0023 | 0.0060 | 0.0004 | 0.0011 | 0.0000 | 0.17 | 0.66 | 0.88 | 0.23 | 0.57 | 0.76 | 0.24 |
| f_Erysipelotrichaceae | 0.0074 | 0.0160 | 0.0015 | 0.0023 | 0.0000 | 0.21 | 0.64 | 0.65 | 0.46 | 0.65 | 0.70 | 0.47 |
| f_S24-7 | 0.0048 | 0.0114 | 0.0011 | 0.0031 | 0.0000 | 0.23 | 0.63 | 0.95 | 0.11 | 0.67 | 0.94 | 0.12 |
| f_Comamonadaceae | 0.0108 | 0.0189 | 0.0029 | 0.0058 | 0.0000 | 0.27 | 0.70 | 0.69 | 0.46 | 0.69 | 0.70 | 0.57 |
| f_Pseudomonadaceae | 0.0652 | 0.0747 | 0.0190 | 0.0277 | 0.0000 | 0.29 | 0.75 | 0.67 | 0.62 | 0.76 | 0.74 | 0.67 |
| f_Rhodobacteraceae | 0.0071 | 0.0199 | 0.0023 | 0.0038 | 0.0002 | 0.33 | 0.64 | 0.89 | 0.16 | 0.60 | 0.83 | 0.16 |
| f_Methylobacteriaceae | 0.0048 | 0.0084 | 0.0016 | 0.0028 | 0.0000 | 0.34 | 0.63 | 0.90 | 0.22 | 0.60 | 0.89 | 0.16 |
| f_Clostridiaceae | 0.0176 | 0.0455 | 0.0067 | 0.0088 | 0.0001 | 0.38 | 0.57 | 0.97 | 0.03 | 0.67 | 0.99 | 0.04 |
| f_Bifidobacteriaceae | 0.0159 | 0.0206 | 0.0064 | 0.0084 | 0.0000 | 0.41 | 0.68 | 0.73 | 0.42 | 0.70 | 0.75 | 0.53 |
| f_Aerococcaceae | 0.0046 | 0.0081 | 0.0020 | 0.0027 | 0.0000 | 0.45 | 0.58 | 0.93 | 0.07 | 0.60 | 0.95 | 0.08 |
| f_[Weeksellaceae] | 0.0047 | 0.0103 | 0.0021 | 0.0098 | 0.0079 | 0.45 | 0.63 | 0.83 | 0.26 | 0.57 | 0.77 | 0.22 |
| f_Veillonellaceae | 0.0129 | 0.0194 | 0.0060 | 0.0086 | 0.0000 | 0.47 | 0.63 | 0.88 | 0.15 | 0.64 | 0.86 | 0.14 |
| f_Carnobacteriaceae | 0.0013 | 0.0034 | 0.0006 | 0.0014 | 0.0037 | 0.47 | 0.77 | 0.96 | 0.44 | 0.65 | 0.96 | 0.33 |
| f_Planococcaceae | 0.0023 | 0.0037 | 0.0011 | 0.0018 | 0.0000 | 0.48 | 0.63 | 0.91 | 0.16 | 0.55 | 0.93 | 0.18 |
| f_Prevotellaceae | 0.0193 | 0.0365 | 0.0093 | 0.0126 | 0.0000 | 0.48 | 0.61 | 0.91 | 0.10 | 0.60 | 0.89 | 0.14 |
| f_Verrucomicrobiaceae | 0.0289 | 0.0443 | 0.0831 | 0.0516 | 0.0000 | 2.87 | 0.88 | 0.92 | 0.62 | 0.78 | 0.86 | 0.57 |
| f_mitochondria | 0.0014 | 0.0053 | 0.0047 | 0.0085 | 0.0000 | 3.26 | 0.69 | 0.93 | 0.25 | 0.65 | 0.94 | 0.22 |
| f_Deferribacteraceae | 0.0020 | 0.0067 | 0.0067 | 0.0068 | 0.0000 | 3.39 | 0.82 | 0.90 | 0.42 | 0.71 | 0.92 | 0.29 |
| f_Peptococcaceae | 0.0010 | 0.0027 | 0.0036 | 0.0045 | 0.0000 | 3.60 | 0.73 | 0.92 | 0.42 | 0.66 | 0.87 | 0.35 |
| f_[Fimbriimonadaceae] | 0.0010 | 0.0041 | 0.0039 | 0.0072 | 0.0000 | 3.94 | 0.60 | 0.96 | 0.21 | 0.67 | 0.98 | 0.18 |
| f_Christensenellaceae | 0.0006 | 0.0016 | 0.0026 | 0.0077 | 0.0009 | 4.22 | 0.61 | 0.95 | 0.10 | 0.65 | 0.99 | 0.06 |
| f_Halomonadaceae | 0.0038 | 0.0075 | 0.0198 | 0.0443 | 0.0000 | 5.25 | 0.82 | 0.89 | 0.50 | 0.78 | 0.88 | 0.41 |
| f_Gordoniaceae | 0.0003 | 0.0011 | 0.0018 | 0.0034 | 0.0000 | 5.67 | 0.62 | 0.94 | 0.24 | 0.64 | 0.89 | 0.29 |
| f_Pseudonocardiaceae | 0.0004 | 0.0012 | 0.0036 | 0.0054 | 0.0000 | 10.22 | 0.73 | 0.95 | 0.43 | 0.66 | 0.90 | 0.39 |
| f_Bdellovibrionaceae | 0.0000 | 0.0002 | 0.0018 | 0.0036 | 0.0000 | 45.44 | 0.64 | 0.97 | 0.28 | 0.63 | 1.00 | 0.27 |

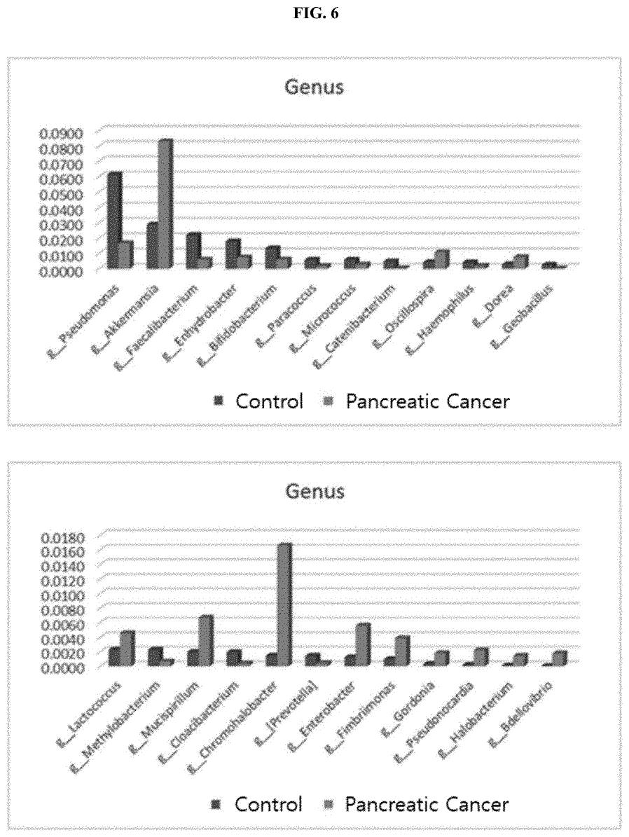

As a result of analyzing bacteria-derived EVs in blood at a genus level, a diagnostic model developed using bacteria belonging to the genus Catenibacterium, the genus Geobacillus, the genus Cloacibacterium, the genus Faecalibacterium, the genus Pseudomonas, the genus Methylobacterium, the genus Prevotella, the genus Paracoccus, the genus Enhydrobacter, the genus Bifidobacterium, the genus Haemophilus, the genus Micrococcus, the genus Lactococcus, the genus Oscillospira, the genus Dorea, the genus Akkermansia, the genus Mucispirillum, the genus Fimbriimonas, the genus Enterobacter, the genus Gordonia, the genus Chromohalobacter, the genus Pseudonocardia, the genus Halobacterium, and the genus Bdellovibrio as a biomarker exhibited significant diagnostic performance for pancreatic cancer (see Table 6 and FIG. 6).

TABLE 6

| | | | Pancreatic | | t-test | | | | | | | |
|---|---|---|---|---|---|---|---|---|---|---|---|---|
| | Control | | Cancer | | p- | | Training Set | | | Test Set | | |
| Taxon | Mean | SD | Mean | SD | value | Ratio | AUC | sensitivity | specificity | AUC | sensitivity | specificity |
| g_Catenibacterium | 0.0049 | 0.0143 | 0.0004 | 0.0012 | 0.0000 | 0.08 | 0.60 | 0.96 | 0.09 | 0.49 | 0.92 | 0.04 |
| g_Geobacillus | 0.0026 | 0.0078 | 0.0004 | 0.0009 | 0.0000 | 0.13 | 0.58 | 0.98 | 0.04 | 0.59 | 0.98 | 0.02 |
| g_Cloacibacterium | 0.0019 | 0.0087 | 0.0004 | 0.0011 | 0.0041 | 0.20 | 0.65 | 0.96 | 0.31 | 0.62 | 0.98 | 0.37 |
| g_Faecalibacterium | 0.0221 | 0.0282 | 0.0060 | 0.0077 | 0.0000 | 0.27 | 0.69 | 0.65 | 0.62 | 0.67 | 0.57 | 0.65 |
| g_Pseudomonas | 0.0615 | 0.0718 | 0.0167 | 0.0261 | 0.0000 | 0.27 | 0.76 | 0.67 | 0.64 | 0.78 | 0.75 | 0.69 |
| g_Methylobacterium | 0.0023 | 0.0067 | 0.0007 | 0.0018 | 0.0002 | 0.29 | 0.58 | 0.99 | 0.01 | 0.55 | 0.99 | 0.00 |
| g_[Prevotella] | 0.0014 | 0.0036 | 0.0004 | 0.0011 | 0.0000 | 0.32 | 0.77 | 0.96 | 0.44 | 0.65 | 0.96 | 0.33 |
| g_Paracoccus | 0.0059 | 0.0194 | 0.0019 | 0.0036 | 0.0010 | 0.32 | 0.61 | 0.98 | 0.02 | 0.57 | 0.98 | 0.02 |
| g_Enhydrobacter | 0.0179 | 0.0228 | 0.0074 | 0.0075 | 0.0000 | 0.41 | 0.64 | 0.72 | 0.42 | 0.61 | 0.74 | 0.33 |
| g_Bifidobacterium | 0.0133 | 0.0177 | 0.0061 | 0.0083 | 0.0000 | 0.46 | 0.63 | 0.81 | 0.26 | 0.67 | 0.81 | 0.24 |
| g_Haemophilus | 0.0043 | 0.0066 | 0.0020 | 0.0031 | 0.0000 | 0.48 | 0.65 | 0.89 | 0.17 | 0.53 | 0.86 | 0.12 |
| g_Micrococcus | 0.0059 | 0.0101 | 0.0029 | 0.0042 | 0.0000 | 0.49 | 0.58 | 0.99 | 0.00 | 0.52 | 0.99 | 0.00 |
| g_Lactococcus | 0.0023 | 0.0048 | 0.0045 | 0.0057 | 0.0000 | 2.00 | 0.81 | 0.94 | 0.30 | 0.78 | 0.98 | 0.29 |
| g_Oscillospira | 0.0044 | 0.0066 | 0.0107 | 0.0097 | 0.0000 | 2.44 | 0.82 | 0.90 | 0.51 | 0.68 | 0.81 | 0.43 |
| g_Dorea | 0.0030 | 0.0052 | 0.0077 | 0.0080 | 0.0001 | 2.53 | 0.58 | 0.95 | 0.09 | 0.51 | 0.99 | 0.06 |
| g_Akkermansia | 0.0289 | 0.0443 | 0.0831 | 0.0516 | 0.0000 | 2.88 | 0.88 | 0.92 | 0.62 | 0.78 | 0.86 | 0.59 |
| g_Mucispirillum | 0.0020 | 0.0067 | 0.0067 | 0.0068 | 0.0000 | 3.39 | 0.57 | 0.94 | 0.14 | 0.54 | 0.96 | 0.14 |
| g_Fimbriimonas | 0.0010 | 0.0040 | 0.0038 | 0.0072 | 0.0000 | 3.94 | 0.60 | 0.96 | 0.21 | 0.67 | 0.98 | 0.18 |
| g_Enterobacter | 0.0012 | 0.0052 | 0.0056 | 0.0127 | 0.0000 | 4.56 | 0.64 | 0.92 | 0.21 | 0.60 | 0.95 | 0.14 |
| g_Gordonia | 0.0003 | 0.0011 | 0.0018 | 0.0034 | 0.0004 | 5.66 | 0.61 | 0.94 | 0.24 | 0.64 | 0.89 | 0.29 |
| g_Chromohalobacter | 0.0015 | 0.0048 | 0.0166 | 0.0435 | 0.0000 | 11.38 | 0.87 | 0.93 | 0.55 | 0.84 | 0.92 | 0.49 |
| g_Pseudonocardia | 0.0002 | 0.0008 | 0.0022 | 0.0039 | 0.0000 | 12.41 | 0.68 | 0.96 | 0.36 | 0.62 | 0.94 | 0.31 |
| g_Halobacterium | 0.0001 | 0.0008 | 0.0014 | 0.0038 | 0.0000 | 15.17 | 0.68 | 0.97 | 0.33 | 0.61 | 0.96 | 0.33 |
| g_Bdellovibrio | 0.0000 | 0.0002 | 0.0018 | 0.0036 | 0.0000 | 58.58 | 0.64 | 0.97 | 0.30 | 0.63 | 1.00 | 0.27 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of providing information for pancreatic cancer diagnosis through bacterial metagenomic analysis, according to the present invention, can be used to predict a risk for pancreatic cancer and diagnose pancreatic cancer by analyzing an increase or decrease in content of extracellular vesicles derived from specific bacteria through bacterial metagenomic analysis using a subject-derived sample.

Extracellular vesicles secreted from bacteria existing in the environment are absorbed into the human body, and thus may directly affect the occurrence of cancer, and it is difficult to diagnose pancreatic cancer early before the onset of symptoms so that efficient treatment thereof is difficult. Thus, according to the present invention, a risk for pancreatic cancer can be predicted through metagenomic analysis of bacteria-derived extracellular vesicles using a human body-derived sample, and thus a risk group of pancreatic cancer can be diagnosed early and predicted, thereby delaying the onset of pancreatic cancer or preventing pancreatic cancer through appropriate management, and even after pancreatic cancer occurs, early diagnosis for pancreatic cancer can be implemented, thereby lowering the incidence of pancreatic cancer and increasing therapeutic effects. In addition, the bacterial metagenomic analysis according to the present invention enables patients diagnosed with pancreatic cancer to avoid exposure to causative factors predicted thereby, whereby the progression of pancreatic cancer is ameliorated, or the recurrence of pancreatic cancer can be prevented.

1. A method of providing information for pancreatic cancer diagnosis, the method comprising:
(a) extracting DNA from extracellular vesicles isolated from a subject sample;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

2. The method of claim 1, wherein process (c) comprises comparing an increase or decrease in content of extracellular vesicles derived from one or more bacteria selected from the group consisting of the phylum Fusobacteria, the phylum Thermi, the phylum Cyanobacteria, the phylum Verrucomicrobia, the phylum Deferribacteres, the phylum Armatimonadetes, and the phylum Euryarchaeota.

3. The method of claim 1, wherein process (c) comprises comparing an increase or decrease in content of extracellular vesicles derived from one or more bacteria selected from the group consisting of the class Erysipelotrichi, the class Betaproteobacteria, the class Deltaproteobacteria, the class Chloroplast, the class Verrucomicrobiae, the class Deferribacteres, the class Fimbriimonadia, and the class Halobacteria.

4. The method of claim 1, wherein process (c) comprises comparing an increase or decrease in content of extracellular vesicles derived from one or more bacteria selected from the group consisting of the order Erysipelotrichales, the order Rhizobiales, the order Burkholderiales, the order Fusobacteriales, the order Deinococcales, the order Rhodobacterales, the order Bifidobacteriales, the order Flavobacteriales, the order Streptophyta, the order Verrucomicrobiales, the order Rickettsiales, the order Deferribacterales, the order Fimbriimonadales, the order Oceanospirillales, the order Anaeroplasmatales, the order Halobacteriales, the order RF32, and the order Bdellovibrionales.

5. The method of claim 1, wherein process (c) comprises comparing an increase or decrease in content of extracellular vesicles derived from one or more bacteria selected from the group consisting of the family Rhizobiaceae, the family Oxalobacteraceae, the family Rikenellaceae, the family Erysipelotrichaceae, the family S24-7, the family Comamonadaceae, the family Pseudomonadaceae, the family Rhodobacteraceae, the family Methylobacteriaceae, the family Clostridiaceae, the family Bifidobacteriaceae, the family Aerococcaceae, the family Weeksellaceae, the family Veillonellaceae, the family Carnobacteriaceae, the family Planococcaceae, the family Prevotellaceae, the family Verrucomicrobiaceae, the family mitochondria, the family Deferribacteraceae, the family Peptococcaceae, the family Fimbriimonadaceae, the family Christensenellaceae, the family Halomonadaceae, the family Gordoniaceae, the family Pseudonocardiaceae, and the family Bdellovibrionaceae.

6. The method of claim 1, wherein process (c) comprises comparing an increase or decrease in content of extracellular vesicles derived from one or more bacteria selected from the group consisting of the genus Catenibacterium, the genus Geobacillus, the genus Cloacibacterium, the genus Faecalibacterium, the genus Pseudomonas, the genus Methylobacterium, the genus Prevotella, the genus Paracoccus, the genus Enhydrobacter, the genus Bifidobacterium, the genus Haemophilus, the genus Micrococcus, the genus Lactococcus, the genus Oscillospira, the genus Dorea, the genus Akkermansia, the genus Mucispirillum, the genus Fimbriimonas, the genus Enterobacter, the genus Gordonia, the genus Chromohalobacter, the genus Pseudonocardia, the genus Halobacterium, and the genus Bdellovibrio.

7. The method of claim 1, wherein the subject sample is blood.

8. The method of claim 7, wherein the blood is whole blood, serum, plasma, or blood mononuclear cells.

9. A method of diagnosing pancreatic cancer, the method comprising:
(a) extracting DNA from extracellular vesicles isolated from a subject sample;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

10. The method of claim 9, wherein process (c) comprises comparing an increase or decrease in content of extracellular vesicles derived from one or more bacteria selected from the group consisting of the phylum Fusobacteria, the phylum Thermi, the phylum Cyanobacteria, the phylum Verrucomicrobia, the phylum Deferribacteres, the phylum Armatimonadetes, and the phylum Euryarchaeota.

11. The method of claim 9, wherein process (c) comprises comparing an increase or decrease in content of extracellular vesicles derived from one or more bacteria selected from the group consisting of the class Erysipelotrichi, the class Betaproteobacteria, the class Deltaproteobacteria, the class Chloroplast, the class Verrucomicrobiae, the class Deferribacteres, the class Fimbriimonadia, and the class Halobacteria.

12. The method of claim 9, wherein process (c) comprises comparing an increase or decrease in content of extracellular vesicles derived from one or more bacteria selected from the group consisting of the order Erysipelotrichales, the order Rhizobiales, the order Burkholderiales, the order Fusobacteriales, the order Deinococcales, the order Rhodobacterales, the order Bifidobacteriales, the order Flavobacteriales, the order Streptophyta, the order Verrucomicrobiales, the order Rickettsiales, the order Deferribacterales, the order Fimbriimonadales, the order Oceanospirillales, the order Anaeroplasmatales, the order Halobacteriales, the order RF32, and the order Bdellovibrionales.

13. The method of claim 9, wherein process (c) comprises comparing an increase or decrease in content of extracellular vesicles derived from one or more bacteria selected from the group consisting of the family Rhizobiaceae, the family Oxalobacteraceae, the family Rikenellaceae, the family Erysipelotrichaceae, the family S24-7, the family Comamonadaceae, the family Pseudomonadaceae, the family Rhodobacteraceae, the family Methylobacteriaceae, the family Clostridiaceae, the family Bifidobacteriaceae, the family Aerococcaceae, the family Weeksellaceae, the family Veillonellaceae, the family Carnobacteriaceae, the family Planococcaceae, the family Prevotellaceae, the family Verrucomicrobiaceae, the family mitochondria, the family Deferribacteraceae, the family Peptococcaceae, the family Fimbriimonadaceae, the family Christensenellaceae, the family Halomonadaceae, the family Gordoniaceae, the family Pseudonocardiaceae, and the family Bdellovibrionaceae.

14. The method of claim 9, wherein process (c) comprises comparing an increase or decrease in content of extracellular vesicles derived from one or more bacteria selected from the group consisting of the genus Catenibacterium, the genus Geobacillus, the genus Cloacibacterium, the genus Faecalibacterium, the genus Pseudomonas, the genus Methylobacterium, the genus Prevotella, the genus Paracoccus, the genus Enhydrobacter, the genus Bifidobacterium, the genus Haemophilus, the genus Micrococcus, the genus Lactococcus, the genus Oscillospira, the genus Dorea, the genus Akkermansia, the genus Mucispirillum, the genus Fimbriimonas, the genus Enterobacter, the genus Gordonia, the genus Chromohalobacter, the genus Pseudonocardia, the genus Halobacterium, and the genus Bdellovibrio.

15. The method of claim 9, wherein the subject sample is blood.

16. The method of claim 15, wherein the blood is whole blood, serum, plasma, or blood mononuclear cells.


SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc